(19) United States
(12) Patent Application Publication
(10) Pub. No.: US 2018/0187228 A1
(43) Pub. Date: Jul. 5, 2018

(54) METHOD FOR THE PRODUCTION OF A LYSATE USED FOR CELL-FREE PROTEIN BIOSYNTHESES

(71) Applicant: RINA-NETZWERK RNA TECHNOLOGIEN GMBH, Berlin (DE)

(72) Inventors: Michael Gerrits, Berlin (DE); Jan Strey, Berlin (DE); Wolfgang Stiege, Berlin (DE)

(21) Appl. No.: 15/700,467

(22) Filed: Sep. 11, 2017

Related U.S. Application Data
(63) Continuation of application No. 14/976,509, filed on Dec. 21, 2015, now abandoned.
Application No. 14/976,509 is a continuation of application No. 14/222,937, filed on Mar. 24, 2014, now abandoned.
Application No. 14/222,937 is a continuation of application No. 13/631,146, filed on Sep. 28, 2012, now abandoned.
Application No. 13/631,146 is a continuation of application No. 10/567,544, filed on Oct. 20, 2008, now abandoned.

(30) Foreign Application Priority Data
Aug. 6, 2013 (DE) 103 36 705.5

(51) Int. Cl. C12P 21/00; C12N 15/70; C12N 1/20; C12P 21/02; C12N 15/67; C12N 1/06

(52) U.S. Cl. CPC C12P 21/00; C12N 15/70; C12N 1/20; C12N 1/06; C12P 21/02; C12N 15/67

(57) ABSTRACT

The invention relates to a method for producing a lysate used for cell-fee protein biosynthesis, comprising the following steps: a) a genomic sequence in an organism, which codes for an essential translation product that reduces the yield of cell-fee protein biosynthesis, is replaced by the foreign DNA located under a suitable regulatory element, said foreign DNA coding for the essential translation product that additionally contains a marker sequence; b) the organism cloned according to step a) is cultivated; c) the organisms from the culture obtained in step b) are lysed; and d) the essential translation product is eliminated by means of a separation process that is selective for the marker sequence. Also discussed are said lysate and the use thereof.

### STATEMENT OF RELATED APPLICATIONS

The present application is a continuation of Ser. No. 14/976,509, filed Dec. 21, 2015, entitled “Method for the Production of a Lysate Used for Cell-Free Protein Biosynthesis,” which is a continuation of Ser. No. 14/222,937, filed Mar. 24, 2014, entitled “Method for the Production of a Lysate Used for Cell-Free Protein Biosynthesis,” which is a continuation of Ser. No. 13/631,146, filed Sep. 28, 2012, entitled “Method for the Production of a Lysate Used for Cell-Free Protein Biosynthesis,” which is a continuation of U.S. patent application Ser. No. 10/567,544, filed Oct. 20, 2008, now abandoned, entitled “Method for the Production of a Lysate Used for Cell-Free Protein Biosynthesis,” which is a 35 U.S.C. 371 National Stage Application of International Application No. PCT/EP04/08469, filed Jul. 27, 2004, which claims priority under 35 U.S.C. § 119(a) to German Patent Application No. 10336705.5, filed Aug. 6, 2003, each of which are incorporated herein by reference in there entireties.

### FIELD OF THE INVENTION

The invention relates to a method for the production of a lysate and to the use of the lysate, wherein the lysate has a low activity of essential translation products, used for cell-free protein biosynthesis of synthetic proteins.

### BACKGROUND OF THE INVENTION

Proteins in high purities, in particular however also in high quantities are needed for biotechnological and medical applications. In most cases, a classic synthesis is not possible, at any case not economical. This relates in particular to the production of modified proteins or of proteins containing non-natural amino acids.

One possibility for the production of proteins in a larger scale is the genetic production. For this purpose, the cloned DNA coding for the desired protein is introduced in cells, in particular prokaryotic cells as a foreign DNA in the form of vectors or plasmids. These cells are then cultivated, and the proteins coded by the foreign DNA are expressed and extracted. In this way higher quantities of proteins can be obtained, however the measures known up to now, in particular cloning, are expensive. Further, the cells are in most cases only transiently transfected and only in exceptional cases stably immortalized. Furthermore, the in vitro protein biosynthesis has several drawbacks: the cell-own expression system suppresses the expression of heterologous gene structures, or respective mRNA or gene products are instable or are destroyed by intracellular nucleases or proteases. For toxic end products, the expression leads to an inhibition or even to the death of the organism, thereby making it difficult for an over-production of the desired protein.

The cell-free protein biosynthesis is an efficient alternative for the synthesis of proteins by genetically modified organisms, since herein the above phenomena are avoidable. Known cell-free protein biosynthesis systems are lysates of rabbit reticulocytes, from wheat sprouts, and bacterial S30 extracts. Methods for the production of a lysate are well known to the man skilled in the art. It continues being problematic, however, when using a lysate wherein the lysate may contain components, which undesirably affect the production of the desired protein and thus reduce the yield. The negative effect of such components may be eliminated by the inhibition or removal thereof from the lysate. Undesired activities when producing lysates are eliminated when that the content of the cell during the processing of the components for the protein biosynthesis is fractioned. During this process for instance, membrane and cell wall components, a large portion of the chromosomal DNA and low-molecular components are separated. Remaining activities have to be removed in further processing steps or prevented beforehand by genetic modification of the organism.

From the document U.S. Pat. No. 6,337,191, the use of a lysate for the production of proteins with an improved energy regeneration system, in which as, an option, disrupting enzyme activities are additionally eliminated by inhibition or removal of the undesired enzymes is known in the art. Potential methods are the knockout method, antisense or further known methods for removing proteins, such as the affinity chromatography.

Further, lysates from genetically modified cell strains are known in the art, which are deficient of certain activities. As an example the genetically modified E. coli strain EcoPro T7 from Novagen is mentioned here, which lacks the proteases Ion and ompT.

In special cases, the protein disrupting the in vitro protein biosynthesis is imperative for the growth of the organism. An inactivation of the enzyme inevitably leads to the death of the organism. In such cases, the enzyme is to be inactivated or removed later. The above-mentioned document U.S. Pat. No. 6,337,191 lists various methods for this purpose.

By the cell-free protein biosynthesis, in particular synthetic proteins comprising unnatural amino acids can be produced. The codon of an amino acid is transformed by mutation into a non-sense codon according to a termination codon. The incorporation of unnatural amino acids is performed by tRNA's being complementary to this termination codon, said tRNA's being synthetically loaded with the unnatural amino acids. The termination codon UAG is the amber codon, accordingly the tRNA's being complementary to the termination codon UAG are called amber-suppressor tRNA's. The incorporation of unnatural amino acids by means of amber-suppressor tRNA's at the UAG stop codon is however in direct competition with the chain termination by the natural termination factor 1 (RF1). Under certain circumstances, the competition is so strong that only a small part of the amino acyl tRNA is used for the protein synthesis, and an undesired large portion of the capacity of the translation system is used for the synthesis of terminated peptides. The consequence of this competitive behavior is a poor incorporation of the unnatural amino acid and thus a lower yield of modified protein, connected with a high number of undesired side products comprising prematurely interrupted or terminated protein chains.

In the document Shimizu et al.; Nature Biotech 19(8):751-755, 1991, a pure system is described, in which a suppressor tRNA efficiently works, if RFI is left away.

From the document Short et al.; Biochemistry 38:8808-8819, 1999, a temperature sensitive termination factor 1 from E. coli is known, which is inactivated by mildly heating the lysate. The increase of the yield with unnatural amino acids of modified proteins is significant. Equally, less side products have to be encountered when producing the protein DHFR. Disadvantageous, in this method, is the heating of the lysate, thereby further thermo sensitive factors of the protein apparatus being destroyed.

### BRIEF DESCRIPTION OF THE DRAWINGS

FIG. 1 shows a diagrammatical representation of the competitive behavior of RF1 and of an amber suppressor tRNA.

FIG. 2 shows the preparative expression and purification of RF1-SII.

FIG. 3 shows the functional test of RF1-SII in the amber suppressor assay. Numeral 1 in FIG. 3A designates the execution of the array in a batch without addition of suppressor tRNA. FIG. 3B shows the tRNA selection rate in dependence on the addition of RF1-SII.

FIG. 4 represents the comparison of the functionality and activity of tagged and of native RF1 in the amber suppression assay. FIG. 4A shows how tRNA selection rates were determined in the presence of both proteins during the expression of the reporter proteins FABPAmb88 from the PhosphoImage. FIG. 4B shows for RF1-SII independence on the added quantity of matrix a smaller synthesis rate than RF1. FIG. 4C shows the tRNA selection rate in the presence of RF1-SII is nearly identical to that in presence of native RF1. Both proteins have thus a comparable activity.

FIG. 5 shows elution behavior of lysate components and in particular of RF1-SII with and without addition of NaCl and the respective share of RF1-SII in the lysate. FIG. 5A shows the elution behavior of lysate components. FIG. 5B shows that RF1-SII specifically binds to the streptactin column and is only eluted by the elution solution from the column. FIGS. 5C1 and 5C2 show the share of RF1-SII in the lysate independence on the respective separation step.

FIG. 6 shows a diagrammatical representation of the chromosomal gene of a protein replaced according to the invention before and after cloning.

FIG. 7A shows the Coomassie staining of the gel. FIG. 7B shows the detection of RF1-SII with streptavidin-HRP on anti-SII (monoclonal antibodies against streptag).

FIG. 8 shows the in vivo expression of RF1-SII in the Western blot.

FIG. 9 represents the result of the in vitro protein biosynthesis with a lysate according to the invention and an RF1-containing lysate. FIG. 9A shows the PhosphoImage of an SDS gel, which shows the respective shares of the termination product and of the suppression product before and after the separation of RF1 in dependence on the quantity of suppressor tRNA. FIG. 9B shows that by the separation of RF1, the translation of the suppression product is increased. FIG. 9C shows that simultaneously the ratio suppression product/termination product is displaced towards the side of the suppression product.

FIG. 10A shows the increased incorporation of biotinyl-lysine in presence of RF1 in FABP. FIG. 10B and FIG. 10C shows that the marking of the translated proteins with ¹⁴Cleucine confirms the higher synthesis rate of biotinylated FABP in an RF1-deficient lysate.

FIG. 11A shows the Western blot. FIG. 11B shows the quantification of the Western blot by the detection of chemiluminescence.

FIG. 12 shows that with a longer reaction time, the yield of the biotinylated suppression product is increased.

### TECHNICAL OBJECT OF THE INVENTION

It is the technical object of the invention to provide a method for the production of a lysate used for cell-free protein biosynthesis, which is simple, and wherein the lysate permits increased yields of synthetic protein in the usual cell-free protein biosynthesis method.

### Definitions

The term “lysate” comprises all active cell extracts produced by the disintegration of eukaryotic or prokaryotic cells.

“Essential translation products” are gene products, which are imperatively needed for the survival and/or proliferation of a cell.

“Synthetic proteins” are proteins produced in a cell-free way.

“Reduced yield” means that the yield of a synthetic protein by cell-free protein biosynthesis in a lysate, which contains the essential translation product, is smaller by 10% related to the weights, preferably 20% to 80%, particularly preferably by more than 90% than the yield of the same synthetic protein in a lysate of the same type and under otherwise identical conditions, from which lysate, however, the essential translation product has been separated.

A “marker sequence” represents a structure, which serves for the identification of molecules, among others of proteins. Such a structure may be a short sequence of amino acids, wherein the number of amino acids preferably is smaller than 10, in particular between 4 and 8. As an example, such a structure is a tag. A marker sequence may also code for enzymes, by means of which the marked molecule can be identified and also separated.

A “selection sequence” codes for a structure, wherein under certain circumstances only the carrier of this selection sequence is permitted to survive. Usually these are resistance genes with regard to certain antibiotics. Further selection sequences may originate from the metabolism of the nucleic acids or of the amino acids.

The term “lysis” designates the dissolution of cells by destruction of the cell wall or cell membrane either under contribution of lytic enzymes or by mechanical or chemical effects.

### Basics of the Invention

For achieving this technical object, the invention teaches a method for the production of a lysate used for cell-free protein biosynthesis, comprising the following steps: a) a genomic sequence in an organism, which codes for an essential translation product that reduces the yield of cell free protein biosynthesis, is replaced by a foreign DNA located under a suitable regulatory element, said foreign DNA coding for the essential translation product that additionally contains a marker sequence; b) the organism cloned according to step a) is cultivated; c) the organisms from the culture obtained in step b) are lysed; and d) the essential translation product is separated from the lysate obtained in step c) by means of a separation process that is selective for the marker sequence. The regulatory element may also be foreign, it may however also be a naturally existing regulatory element. In the first case, the regulatory element must be introduced in the same step as the introduction of the foreign DNA or in a step different thereof.

The production of the lysate according to the invention is simple, and the obtained lysate permits higher yields of synthetic protein in cell-free protein biosynthesis methods, in particular a high yield of proteins with non-natural amino acids.

This is achieved when the essential translation product is provided with a marker sequence, by means of which the essential translation product is removed from the lysate or inhibited in its activity because of the affinity of the marker sequence. The modification of the essential translation product is performed in the chromosomal gene of the protein, such that the essential translation product is expressed in fusion with the marker sequence. A marker sequence codes for a structure, which has a high affinity for (in most cases immobilized) binding sites in separation systems for the purification or to inhibitors. Thereby the activity of the essential translation product can be removed from a mixture of proteins or a mixture of arbitrary molecules, which do not contain the marker sequence.

By the incorporation of the marker sequence in the chromosomal gene of the essential translation product of the organism is achieved by a stable transformation of the organism. Under this condition, a cultivation of the genetically modified organism is possible without a loss of its additional genetic information, and without selection pressure.

A preferred feature of the present invention is that the marker sequence does not affect the protein properties of the essential translation product. An active essential translation product is advantageous for a successful cultivation of the genetically modified organism. The determination of the functionality of the essential translation product provided with a marker sequence takes place by an assay being specific for the function of the essential translation product. For this purpose, a DNA fragment coding for the essential translation product and the marker sequence is translated by an expression PCR. The functionality is evaluated on the basis of the synthesis rate of the product, in the synthesis of which the essential translation product is involved. The synthesis rate in presence of the native essential translation product is compared to the synthesis rate in presence of the modified essential translation product, and the functionality is thus evaluated. The functionality of the essential translation product is not affected by the marker sequence, if the product synthesis rate of the marked essential translation product is 10%, preferably 40 to 60%, in particular over 90% of the synthesis rate of the native essential translation product.

The lysate from a stably transformed organism according to the invention contains up to 100% w/w (referred to the total amount of translation product) of the essential translation product in fusion with the marker sequence and is contaminated, if at all, only slightly (<10% w/w, even <1% w/w, referred to the total amount of translation product) with the natural essential translation product. By the marker sequence, the essential translation product undesired in the lysate can easily and efficiently by removed from the lysate. Consequently, the protein biosynthesis of synthetic proteins, in which non-natural amino acids are incorporated, can be performed more quickly, at higher yields and with a smaller number of side products.

Another advantage of the invention is that only one undesired component can specifically be removed from the lysate. It may however also be possible that several undesired translation products are provided with different marker sequences, advantageously however with the same marker sequence, such that all undesired translation products can be removed by using one separation method. Insofar, the step a) of the method can be performed for different translation products, and the marker sequences may respectively be identical or different.

### Embodiments of the Invention

The cloning of the organism can be performed by transformation methods well known to those skilled in the art, such as microinjection, electroporation, or by chemically mediated receptions of the DNA.

The isolation of the successfully cloned organism is performed by using the selection sequence according to methods known to those skilled in the art.

The cultivation of the organism may be performed in a batch, fed-batch or continuous method.

Equally, the protein biosynthesis of synthetic proteins comprising non-natural amino acids may be performed in a batch, fed-batch or continuous method.

The lysis of the cells takes for instance place by mechanical action such as high-pressure homogenization, by ultrasound or by decomposition in ball mills.

In another preferred embodiment, the essential translation product is the termination factor RF1, which detects the termination codon UAG. It is understood that the essential translation product can also be selected from other proteins, which reduce or disrupt the function of a lysate for the cell-free protein biosynthesis. For instance, the essential translation product may be another termination factor or a protein, which interacts with a termination factor, for instance HemK. Other factors of the translation, the inactivation of which would be lethal for the living cell, the removal of which however exerts a positive influence on the efficiency of the translation or other applications of the lysate, can for the purpose of the invention be removed from the lysates. For instance, the essential translation product may comprise an amino acyl tRNA synthetase, the removal of which would lead to an inactivation of the respective tRNA's detected by the synthetase, such that at last a certain amino acid can be replaced by another one at selected codons. In this context is preferred the cysteinyl tRNA synthetase, by the removal of which from the lysate the two codons for cysteine would be available for other unnatural or modified-amino acids. In principle, all amino acyl tRNA synthetases, in particular those, which relatively rarely activate amino acids contained in proteins, are imaginable. Another essential translation product is the methionyl tRNA transformylase catalyzing the formylation of the prokaryoticmethionyl initiator tRNA (Met-tRNAf). The removal of this enzyme- or also of another enzyme of the formylation pathway from a system for the cell-free protein biosynthesis would essentially reduce or even completely eliminate the translation initiation with natural methionine. Thereby the efficiency of initiator tRNA's, which have been preacylated with N-formylated modified or unnatural amino acids, for instance fluorescent or biotinylated amino acids, could considerably be increased, and thus the synthesis of cotranslationally N-terminally modified proteins could enormously be raised. The marking degree of such modified proteins could also substantially be increased, probably up to nearly 100%. Another possibility is to take an initiation factor from the system, in order to specifically intervene in the initiation. For instance, this factor could then be given back into the system, together with preacylated tRNA, or be replaced by another initiation factor. Other examples for essential translation products can be selected from the group of the phosphatases and for instance positively influence the energy consumption of the lysates. The manipulation of enzymes of the amino acid metabolism, for instance the removal of amino acid transferases or isomerases, is suitable for permitting the introduction of individual marked amino acid species without scrambling. Of course, essential translation products may also be selected from the group of eukaryotic proteins. For instance named factors of the eukaryotic translation inhibitors, such as eIF2. This factor has a regulatory sub-unit, eIF2α, which inhibits in its phosphorylated form the initiation of the translation. Since eIF2 is also active without this sub-unit, the removal of eIF2α would lead to an improvement of the translation initiation and thus to an improvement of the protein yields in the eukaryotic cell-free system. Factors from the group of the nucleases, proteases, kinases, racemases, isomerases, dehydrogenases or polymerases may also be preferred targets of the prokaryotic or eukaryotic system.

In a particular embodiment, the marker sequence is selected from the group “streptag-II, polyhistidine, FLAG, polyarginine, polyaspartate, polyglutamine, polyphenylalanine, polycystin, Myc, gluthathione S-transferase, protein A, maltose-binding protein, galactose-binding protein, chloramphenicolacetyl transferase”. Further examples are mentioned in the patent claims.

The marker sequence and the chromosomal gene of the essential translation product are expressed as a fusion protein. In a preferred embodiment, the marker sequence is a streptag-II, a peptide structure of 8 amino acids with affinity to streptactin. For instance, the expressed termination factor RF1 may comprise the streptag-II at the c-terminal end. The separation of the RF1-streptag-II fusion protein is performed correspondingly at an affinity matrix loaded with streptactin or other SII-binding matrices. The separation may be performed on the basis of column-chromatographic methods, but also by batch methods. It is understood that another marker sequence and its respective affinity partner may also be used. An example is the poly-His tag. A poly-His tag normally consists of six successive histidine residues, which may however have a length between 4 and 10 residues. In another preferred embodiment, the isolation of the essential translation products is performed by corresponding antibodies, antibody fragments or by aptamers. Under certain circumstances, the affinity of the binding partners also causes a simultaneous inhibition of the activity of the essential translation product.

With regard to the selection of the method for protein separation, a method is to be selected, which does not affect the translation activity of the lysate, i.e. does not separate important reaction components of the translation system.

In principle, the organism may be a eukaryote or aprokaryote. It is particularly helpful, if the organism for the production of a lysate is a prokaryote. With regard thereto, reference is made to the patent claims. Particularly suitable is the translation system from Escherichia coli.

The invention further teaches a lysate for the cell-free protein biosynthesis having a reduced activity of an essential translation product and the use thereof for the production of synthetic proteins comprising non-natural or modified natural amino acids. In a preferred embodiment, the lysate comprises a reduced activity of a factor involved in the termination, preferably RF1. An example for the production of modified synthetic proteins is the incorporation of biotinyllysine (biocytin) by means of an amber-suppressor tRNA aminoacylated with the amino acid. With regard to the synthesis and purification of biotinylated or other streptactin binding proteins, the system has another advantage: Since endogenous biotinylated proteins are also separated during the separation of RF1-II, a contamination of synthetic proteins, which are purified by means of streptavidin or similar matrices, with biotinylated proteins from the production strain is prevented. The lysate also permits however the more efficient incorporation of other functional groups in proteins, particularly preferred the incorporation of fluorophores, or that of a universally reactive group, by which other functions can selectively and position-specifically be coupled. An alternative of use is also the incorporation of natural amino acids, which may be present for instance in an isotope-marked or selenium-containing structure.

The loading of the amber suppressor tRNA with the unnatural amino acid can be performed with the so-called chemical aminoacylation or also by means of enzymes, for instance synthetases or ribozymes. It is also possible to combine enzymatic and different chemical methods with each other. For instance, the tRNA can first be aminoacylated chemically or enzymatically with lysine, cysteine or another amino acid containing a reactive function in the side chain. Then, to the corresponding amino acyl tRNA, via the reactive function of the amino acid, an interesting functional group, for instance a fluorophore, is coupled by using conventional chemical methods. For instance, the sulfhydryle group of cysteine can be modified by maleimide, or an amino group by an NHS ester. The amino acyl binding of the tRNA can be stabilized during the modification, for instance by the presence of a protective group at the alpha amino group.

The system is suitable for answering and solving scientific questions of the protein research. Further, the system is in principle suitable for a ribosome display, since after removal of a termination factor the respective codon cannot be read, and thus the ribosomal complex of mRNA, synthetic protein and ribosome has an increased stability. The system also permits a defined introduction of puromycin or respective derivatives at the position of the above-mentioned codon. Puromycin normally competes with the ternary complex or termination factors and is statistically added to the end of the growing protein chain. The generation of a “starved” codon by the removal of a termination factor permits the defined incorporation of puromycin at this position. In this way, functions can be appended to synthetic proteins, said functions being coupled to the puromycin, for instance DNA oligomers, sugar or other components.

In another embodiment, the lysate may also have a reduced activity of another essential translation product, for instance one of the group of the phosphatases, the nucleases, the synthetases or proteases. Thereby the production of such synthetic proteins can be improved, the synthesis of which is limited by the activity of other essential translation products than by the activity of the termination factors.

It is also possible, by means of the disclosed method, to remove certain essential translation products from the lysate, which disrupt the answers to certain scientific questions, or the removal of which permits an investigation of certain questions.

In the following, the invention is explained in more detail by way of non-limiting examples.

### Example 1: Competitive Behavior of RF1 and Amber Suppressor tRNA

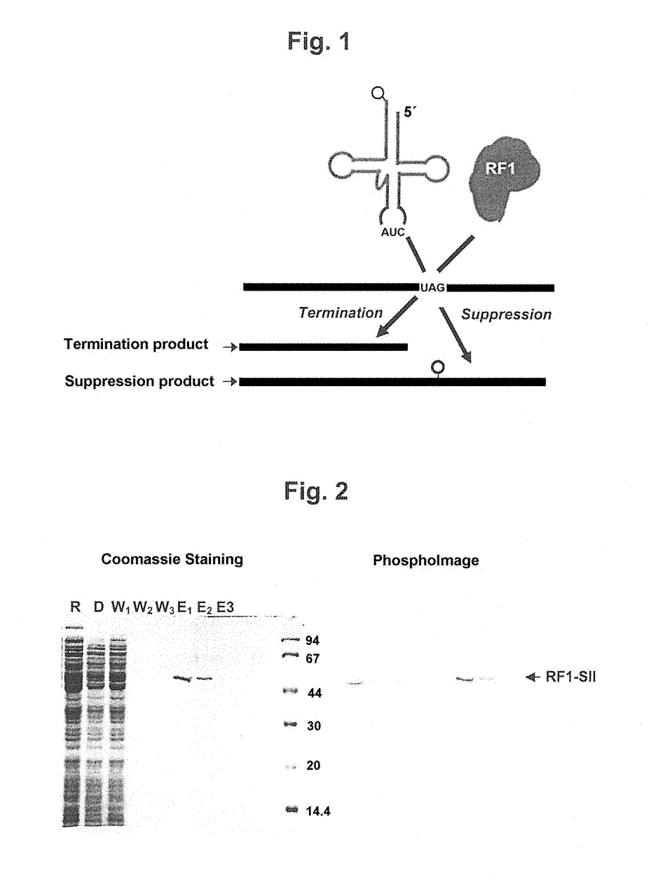

In FIG. 1 there is shown a diagrammatical representation of the competitive behavior of RFI and of an amber suppressor tRNA. Depending upon which of the two molecules pairs with the codon UAG, the protein is terminated or incorporated in an amino acid, and the translation is continued by forming the suppression product.

### Example 2: Pre-Investigations of the Functionality of RF1-SII: Expression PCR

Since an inactivation of the termination factor RF1 would be lethal for the organism, the influence of the appended streptag II on the activity of RF1 was investigated. For this investigation, RF1 was translated exclusively of expression PCR products. FIG. 2 shows the preparative expression and purification of RF1-SII. R represents the in vitro translation reaction, D the run number, W1, W2, W3 the wash fractions and E1, E2, E3 the elution fraction.

### Example 3: Pre-Investigations of the Functionality of RF1-SII: Amber Suppressor Assay

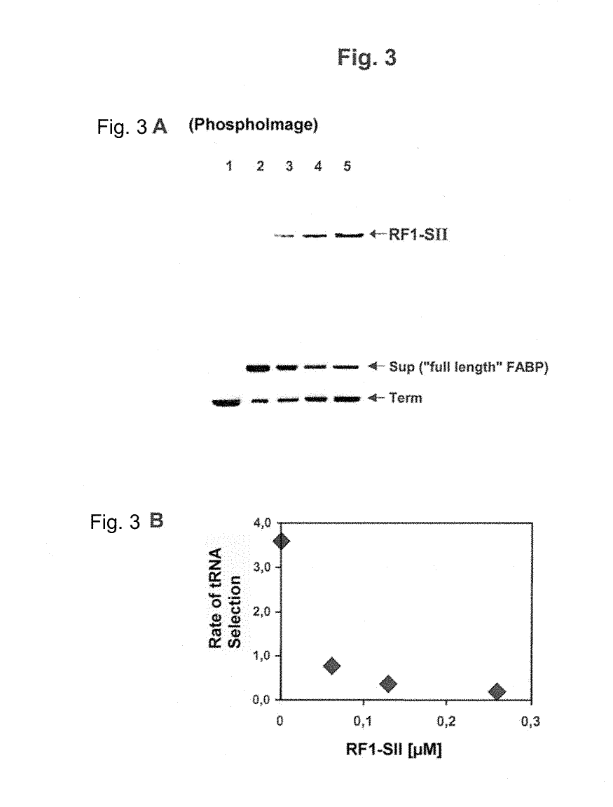

FIG. 3 shows the functional test of RF1-SII in the amber suppressor assay. The numeral 1 in FIG. 3A designates the execution of the array in a batch without addition of suppressor tRNA. The numerals 2 to 5 are batches with suppressor tRNA (1 μM). Batch 2 does not contain any RF1-SII. The batches 3 to 5 are enriched with purified RF1-SII (3: 0.0625 μM, 4: 0.13 flM, 5: 0.26 μM). FIG. B shows the tRNA selection rate in dependence on the addition of RF1-SII. The “tRNA selection rate” is calculated by determining the molar quantities of synthetic suppression and synthetic termination based on a PhosphoImage, and the ratio of the two values is calculated. The increase of the RF1-SII shares in the batch will lead to an increased production of the termination products. FIG. 3B shows the tRNA selection rate in dependence on the quantities RF1-SII in the batch. The tRNA selection rate drops with the addition of RF1-SII from 3.5 to below 1 and can further be reduced by increasing the RF1-SII share. This confirms that RF1-SII is in principle active.

### Example 4: Pre-Investigations of the Functionality of RF I-SII: Activity Comparison with Native RFI

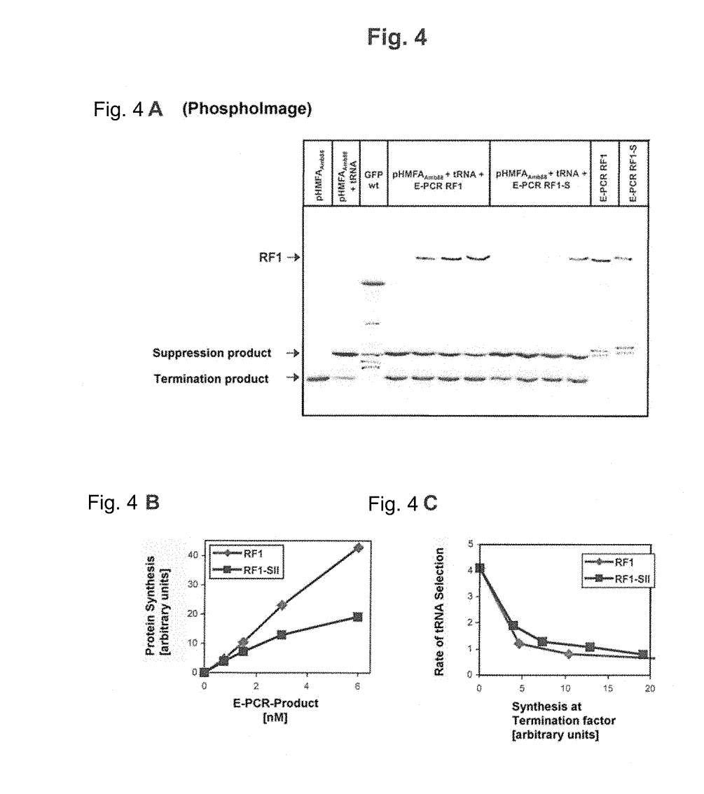

FIG. 4 represents the comparison of the functionality and activity of tagged and of native RF1 in the amber suppression assay. RF1-SII shows a comparable activity as RF1. FIG. 4B shows for RF1-SII independence on the added quantity of matrix a smaller synthesis rate than RF1. Under consideration of the synthesis rates of RF1-SII and native RF 1, then the tRNA selection rates were determined in presence of both proteins during the expression of the reporter proteins FABPAmb88 from the PhosphoImage (FIG. 4A). The matrix (PHMFAAmb88) coding for the reporter protein contains an amber mutation at the amino acid position 88. The tRNA selection rate in presence of RF1-SII is nearly identical to that in presence of native RF1 (diagram 4C). Both proteins have thus a comparable activity.

### Example 5: Simulation of the Removal of RF1-SII from Lysates

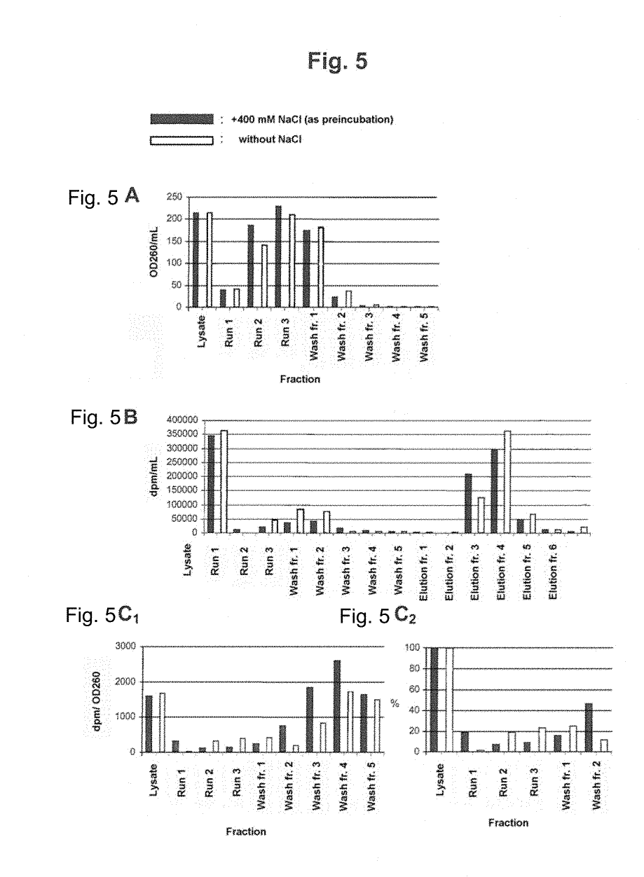

For the simulation of the removal of RF1-SII from lysates, RF1-SII was produced preparatively and marked with ¹⁴C leucine (100 dpm/pmole). Thereafter, the synthesized, purified RF1-SII was added to an S30 lysate in a final concentration of 0.1 μM (in 1×TLM buffer, 215 A₂₆₀/ml). The separation of the RF1-SII is performed by a streptactin column, and in total 500 fll lysate (=approx. 110 A₂₆₀) were applied to 200 μl column in three steps of 166 μl each. The washing volumes were 200 μl each. In FIG. 5 there is shown the elution behavior of lysate components and in particular of RF1-SII with and without addition of NaCl and the respective share of RF1-SII in the lysate. FIG. 5A shows the elution behavior of lysate components. From FIG. 5A can be seen that the lysate components were for the most part not or only non-specifically bound to the column. Non-specifically bound lysate components were slightly eluted again by washing (wash fractions). The employed method thus does not reduce the activity of the lysate by separation of desired lysate components. In FIG. 5B there is shown the elution behavior, and there is disclosed that RF1-SII specifically binds to the streptactin column and is only eluted by the elution solution from the column (elution fraction, FIG. 5B). In the fractions of the run and the wash, RF1 is contained to a small degree only. The FIGS. CI and C2 show the share of RF1-SII in the lysate independence on the respective separation step. FIG. CI contains the values dpm RF1/ml in relation to OD260/ml of the lysate. The share of RF1-SII (dpm/OD260) in the pure lysatein FIG. 5C1 is set to 100% in FIG. 5C2, so that FIG. 5C2 represents the percentage share of RF1-SII in the lysate. FIG. 5C2 shows that RF1-SII is contained in the lysate to a clearly smaller degree after the separation steps “run” and “wash fraction”.

### Example 6: Genomic Structure of a Genetically Modified Organism

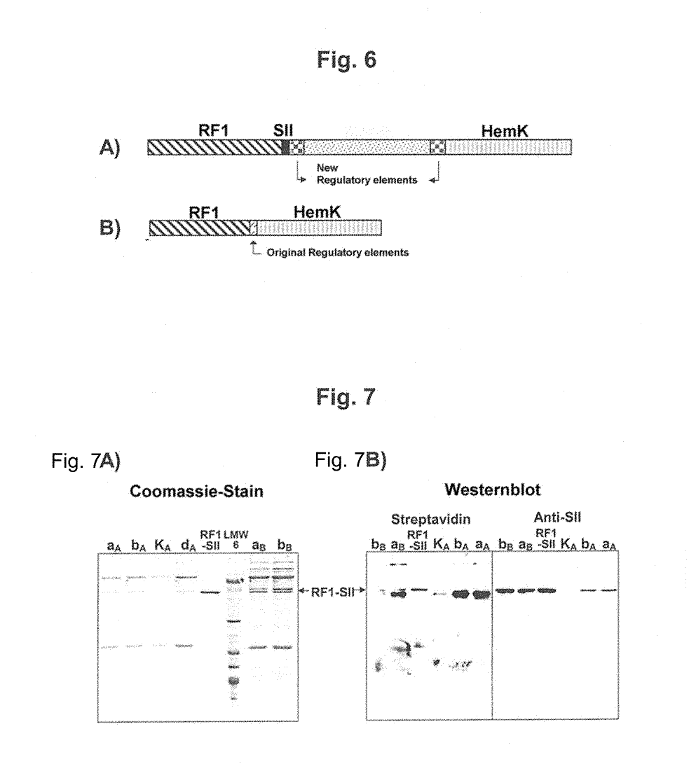

In FIG. 6 there is shown a diagrammatical representation of the chromosomal gene of a protein replaced according to the invention before and after cloning. The original genomic situation (FIG. 6B), which consists of the RF1 gene, a regulatory element and the gene for HemK, and the desired genetic situation (FIG. 6A), where the marker sequence of streptag II is appended to the gene of RF1, can be seen. Furthermore, the desired genetic situation comprises a selection sequence, in this case an antibiotic resistance against kanamycin and new regulatory elements. An organism according to the invention is deposited at the “Deutsche Sammlungvon Mikroorganismen and Zellkulturen GmbH” under the Budapest Treaty, with the number DSM 15756 (E. coli/RF1-SII).

### Example 7: Production of a RF1-Deficient Lysate

The cultivation of three E. coli/RF1-SII clones (a, b, d) was performed in shaken cultures. The cultures were harvested in the log phase and decomposed by means of ultrasound. The RF1-SII containing lysate was divided into two batches, and RF1-SII was separated by different methods. From batch A (in FIG. 5 according to index A) RF1-SII was separated by affinity chromatography at a streptactin column (500 μlysate (=approx. 110 A₂₆₀) on 200 μl column). The batch B (in FIG. 7 according to index B) was subjected to a preincubation (400 mM NaCl) and then RF1-SII was separated over a streptag column (500 μl lysate (=approx. 125A₂₆₀) on 200 μl column). Thereafter the removal of salt by NAP 5 was performed. The results are shown in FIGS. 7 A and B showing the detection of RF1-SII by means of SDS page and Western blot in the elution volume. FIG. 7A shows the Coomassie staining of the gel. FIG. 7B shows the detection of RF1-SII with streptavidin-HRP on anti-SII (monoclonal antibodies against streptag). As a standard serves RF1-SII translated in vitro and purified. LMW6 is a molecular weightmarker, K_{A}a lysate from a genetically unmodified E. coli strain, which was subjected to the separation method of the batch A. The results show that RF1-SII was successfully separated by both methods from the lysate.

### Example 8: Expression of RF I-SII

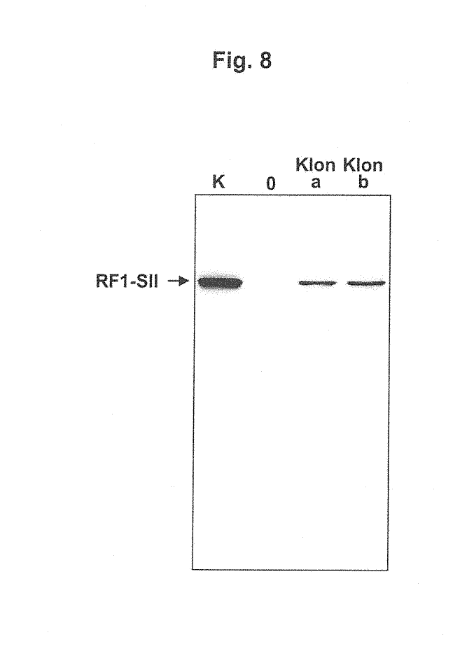

Two E. coli strains were cloned with the synthetic DNA fragment mentioned in Example 6 (desired genetic situation). By means of the expression PCR, the proteins RFI and HemK from the chromosomal DNA (E. coli K12) were amplified, cloned and sequenced. By means of the PCR, the streptag sequence (SII) was added to the gene for RF1, and the new regulatory elements for the expression of HemK were introduced. Both proteins were cell-freely translated, in order to test their expressability and in the case of RF1 also their functionality. Then followed by the production of the gene cassette with the desired genomic situation for the chromosomal replacement. Three PCR fragments (with the genes for RF1-SII, for the kanamycin resistance and for HemK) were produced and ligated with each other. The ligation took place by using asymmetric restriction interfaces in a one-pot reaction, i.e. the three fragments were ligated in one step with each other. The resulting DNA fragment having the desired genomic situation was gel-eluted, cloned in a vector, sequenced and amplified by means of the PCR. Then followed by the transformation of the PCR-generated linear fragment in E. coli D10 by means of the electroporation. The kanamycin resistance was used for the selection for clones having the desired genomic situation. For this purpose, the cells were plated out on kanamycin plates. The four positive clones were subjected to a counter-selection in an ampicillin containing medium, in order to be able to exclude that the plasmid carrying an ampicillin resistance and being used for the amplification of the gene fragment was transformed. Furthermore, the presence of the desired gene fragment within the E. coli chromosome was investigated by means of the colony PCR. For this purpose, a primer hybridizing within the cassette was combined with a primer hybridizing in the E. Coli chromosome outside the transformed cassette. All four clones had the desired genetic situation. FIG. 8 shows the in vivo expression of RF1-SII in the Western blot. The separation of RF1-SII was performed by a streptactin column. The detection of the protein was made with anti-SII (monoclonal antibody against streptag). FIG. 8 shows a clear expression of RF1-SII in the two clones a and b. The negative control “0” from a genetically unmodified strain showed no expression of RF1-SII. The sample “K” is RF1-SII translated in vitro and serves as a marker and positive control.

### Example 9: Influence of the RFI Separation on the Efficiency of the Suppression in the Regenerable System

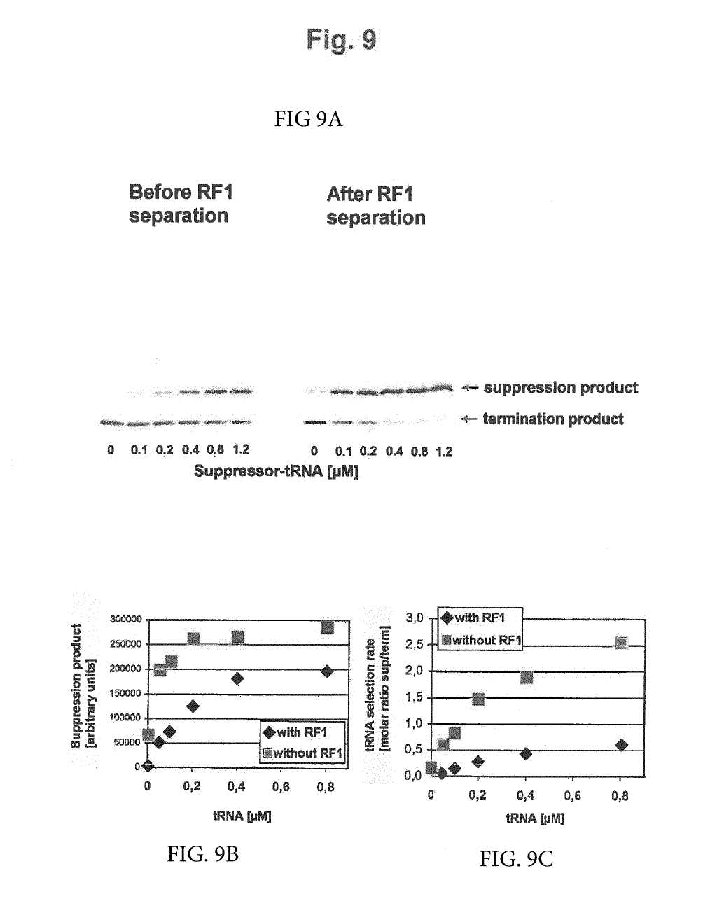

FIG. 9 represents the result of the in vitro protein biosynthesis with a lysate according to the invention and an RF1-containing lysate. FIG. 9A shows the PhosphoImage of an SDS gel, which shows the respective shares of the termination product and of the suppression product before and after the separation of RF 1 in dependence on the quantity of suppressor tRNA. In this case, an enzymatic amino acylatable tRNA was used. With a suppressor tRNA share of 1.2 in the RF1-deficient lysate, small quantities only of the termination product are detectable. By the separation of RF1, the translation of the suppression product is increased (FIG. 9B) and simultaneously the ratio suppression product/termination product is displaced towards the side of the suppression product (FIG. 9C). Furthermore, the synthesis rate of the suppression product is increased by addition of higher quantities of suppressor tRNA. As a result, by separation of RF1 from alysate, the synthesis rate of a suppression product is clearly increased, and the yield is thus also increased.

### Example 10: Incorporation of a Non-Natural Amino Acid

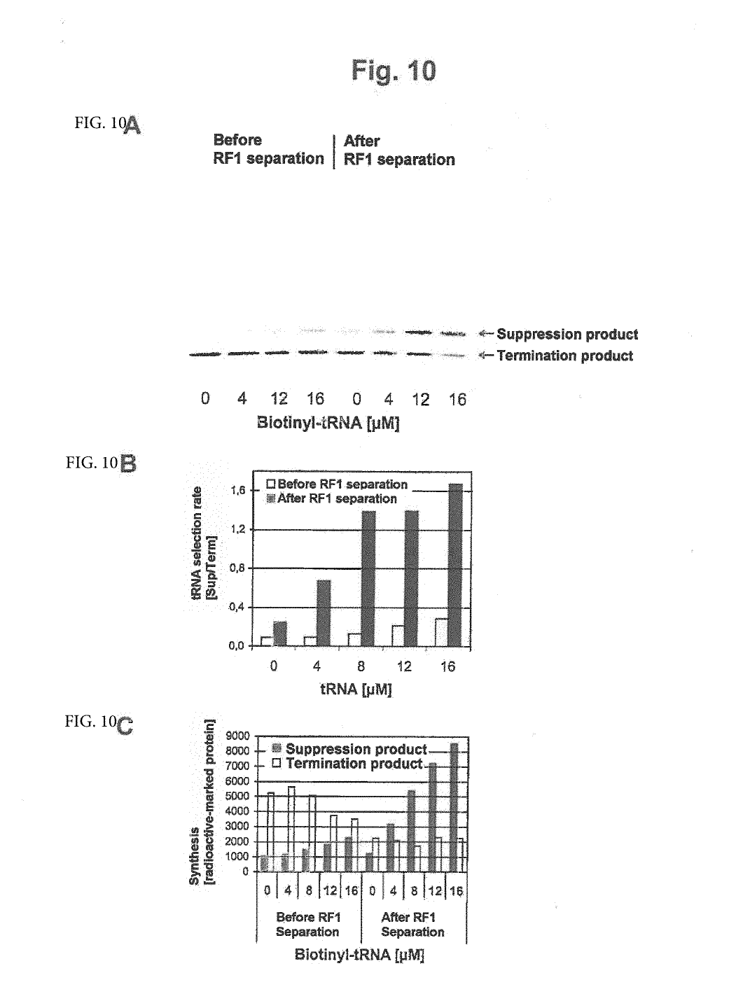

FIG. 10 shows exemplarily the increased incorporation of biotinyl-lysine in presence of RF1 in FABP (FIG. 10A, PhosphoImage). An amber suppressor tRNA is used, which was loaded by chemical methods with biotinyl-lysine (biocytin). The marking of the translated proteins with ¹⁴Cleucine confirms the higher synthesis rate of biotinylated FABP in an RF1-deficient lysate (FIGS. 10B and C).

### Example 11: Incorporation of Biocytin by Means of an Amber Suppressor tRNA Loaded with Chemical Methods Detection of Biotinylated Proteins in the Western Blot

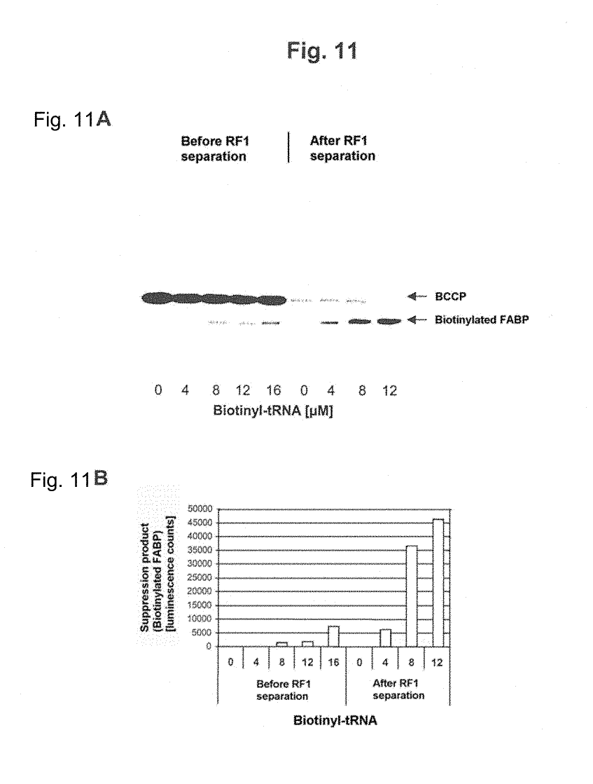

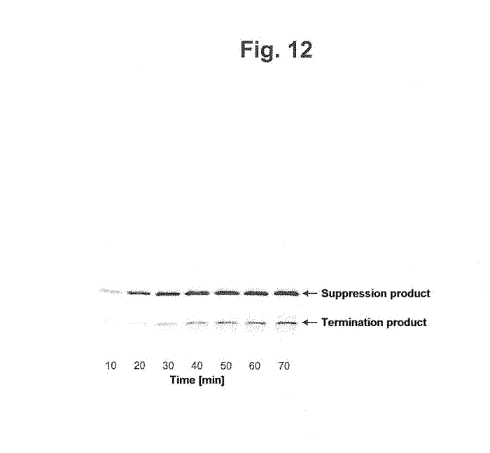

FIG. 11A shows the Western blot, 11B the quantification of the Western blot by the detection of chemiluminescence. A monoclonal antibody against streptag II was used, which was coupled with HRP. The Western blot clearly shows the strongly increased synthesis of synthetic biotinylated protein in the lysate after RF1 separation. Furthermore, the blot shows that by the used method for the production of the RF1-deficient lysate, endogenous biotinylated proteins can also be removed: The endogenous BCCP relatively highly concentrated in lysates of E. coli is nearly not detected anymore after RF1 separation. The quantification of the Western blot once again shows the strongly increased synthesis of synthetic modified protein in the RF1-deficient lysate and confirms the quantification of Example 10 performed by means of the radioactivity.

### Example 12: Incorporation of Biocytin in Dependence on the Reaction Time

With a longer reaction time, biotinyl-lysine (biocytin) is incorporated to a higher degree, as FIG. 12 shows. Consequently, the share of suppression product in the total product quantity grows. By a longer reaction time, the yield of the biotinylated suppression product is increased.

What is claimed is:

1. A method for the production of a lysate used for cell-free protein biosynthesis, comprising the following steps:
a) a genomic sequence in an organism, which codes for an essential translation product that reduces the yield of cell-free protein biosynthesis, is replaced by a foreign DNA located under a suitable regulatory element, said foreign DNA coding for the essential translation product that additionally contains a marker sequence;
b) the transformed organism according to step a) is cultivated;
c) the organisms from the culture obtained in step b) are lysed; and
d) the essential translation product is separated from the lysate obtained in step c) by means of a separation process that is selective for the marker sequence.

2. A method according to claim 1, wherein the essential translation product is selected from the group consisting of termination factors or proteins interacting with termination factors—in particular RF1, RF2, RF3, eRF, L11 or HemK.

3. A method according to claim 1, wherein the marker sequence is selected from the group consisting of streptag II, polyhistidine, FLAG, polyarginine, polyaspartate, polyglutamine, polyphenylalanine, polycysteine, Myc, gluthathione S-transferase, protein A, maltose-binding protein, galactose-binding protein, chloramphenicol acetyl transferase, protein G, calmodulin, calmodulin-binding peptide, HAT (=natural histidine affinity tag), SBP (=streptavidin-binding peptide), chitin-binding domain, thioredoxin, β-galactosidase, S-peptide (residues 1-20 of the Rnase A), avidin, streptavidin, streptag-I, dihydrofolatereductase, lac repressor, cyclomaltodextringlucanotransferase, cellulose-binding domain, btag, nanotag.

4. A method according claim 1, wherein the marker sequence and the chromosomal gene are expressed as a fusion protein, and wherein the translated marker sequence does not affect the activity of the essential translation product in the organism.

5. A method according to claim 1, wherein the separation step is an affinity chromatography or an antibody assay.

6. A method according to claim 1, wherein the organism is a prokaryote or an eurokaryote, in particular selected from the group comprising enterobacteriales (e.g. escherichia spec., E. coli), lactobacillales (e.g. lactococcus spec., streptococcus spec.), actinomycetales (e.g. streptomyces spec., corynebacterium spec.), pseudomonas spec., caulobacter spec., clostridium spec., bacillus spec., thermotoga spec., micrococcus spec., thermus spec.

7. A lysate for the cell-free protein biosynthesis obtainable by a method according to claim 1, wherein the lysate has a reduced activity of an essential translation product.

8. A lysate for the cell-free protein biosynthesis according to claim 7, wherein the lysate has a reduced activity of one or several essential translation products selected from the group consisting of termination factors or proteins interacting with termination factors—in particular RF1, RF2, RF3, eRF, L11 or HemK—, initiation factors or proteins interacting with initiation factors, elongation factors or proteins interacting with elongation factors, aminoacyltRNAsynthetases—in particular cysteinyl tRNA or tryptophanyl tRNA synthetase—, enzymes of the amino acid metabolism—in particular amino acid transferases, isomerases, synthetases phosphatases, nucleases, proteases, kinases, racemases, isomerases, polymerases and combinations of the above substances.

9. A method of using the lysate according to claim 7 for the cell-free protein biosynthesis comprising reducing the activity of an essential translation product.

10. The method for using the lysate according to claim 9 further comprising the step of incorporating amber suppressor tRNA's natural or non-natural amino acids, in particular biotinyl-lysine, fluorescent amino acids and/or phenyl-analine.

11. An isolated microorganism or an isolated cell, wherein a genomic sequence, which codes for an essential translation product that reduces the yield of cell-free protein biosynthesis is replaced by a foreign DNA located under a suitable regulatory element, said foreign DNA coding for the essential translation product that additionally contains a marker sequence.

12. A microorganism, as deposited under DSM 15756.
